(19) United States
(12) Patent Application Publication
(10) Pub. No.: US 2022/0096152 A1
(43) Pub. Date: Mar. 31, 2022

(54) BALLOON CATHETER WITH MICROPOROUS PORTION

(71) Applicant: Boston Scientific Scimed Inc., Maple Grove, MN (US)

(72) Inventors: Edward J. Maierhofer, Brooklyn Park, MN (US); Ryan P. Skarda, Brooklyn Park, MN (US)

(21) Appl. No.: 17/488,219

(22) Filed: Sep. 28, 2021

Related U.S. Application Data
(60) Provisional application No. 63/085,609, filed on Sep. 30, 2020.

(51) Int. Cl. A61B 18/14

(52) U.S. Cl. CPC A61B 18/1492; A61B 2018/00577

(57) ABSTRACT

A catheter for ablation including a catheter shaft, a balloon at a distal end of the catheter shaft, and a microporous portion. The balloon is configured to support conductors and electrodes and contain a fluid. The microporous portion is coupled to the balloon to allow the fluid to flow out of the balloon and includes a plurality of apertures configured to prevent large air bubbles from exiting the balloon.

### CROSS REFERENCE TO RELATED APPLICATION

This application claims priority to Provisional Application No. 63/085,609, filed Sep. 30, 2020, which is herein incorporated by reference in its entirety.

### TECHNICAL FIELD

The present disclosure relates to medical systems and methods for ablating tissue in a patient. More specifically, the present disclosure relates to a balloon catheter useful during an ablation procedure.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. Planning irreversible electroporation ablation procedures can be difficult due to the lack of acute visualization or data indicating which tissues have been irreversibly electroporated, as opposed to reversibly electroporated. Where tissue recovery can occur over minutes, hours, or days after the ablation is completed.

### SUMMARY

As recited in examples, Example 1 is a catheter for ablation. The catheter comprises a catheter shaft, a balloon at a distal end of the catheter shaft, and a microporous portion. The balloon is configured to support conductors and electrodes and is configured for containing a fluid. The microporous portion is coupled to the balloon, configured to allow the fluid to flow out of the balloon, and comprises a plurality of apertures configured to prevent air bubbles with a diameter larger than 50 microns from exiting the balloon.

Example 2 is the catheter of Example 1, wherein the microporous portion forms a portion of the balloon.

Example 3 is the catheter of any one of Examples 1 and 2, wherein the microporous portion of the balloon is a disc-shaped portion disposed at a distal portion of the balloon.

Example 4 is the catheter of any one of Examples 1-3, wherein the balloon includes a microporous strip portion comprising a plurality of apertures.

Example 5 is the catheter of any one of Examples 1-4, wherein the entire balloon is microporous with a plurality of apertures.

Example 6 is the catheter of any one of Examples 1-5, wherein the catheter further comprises a tubular portion coupled to the shaft and to the balloon, wherein the microporous portion is integrated into the tubular portion.

Example 7 is the catheter of any one of Examples 1-6, wherein the plurality of apertures each have a diameter of between 0.05 microns and 50 microns.

Example 8 is the catheter of any one of Examples 1-7, wherein the microporous portion is configured such that at a balloon operating pressure of 1 psi, the fluid exits the balloon at an operating flow rate of less than or equal to 1 ml/min.

Example 9 is the catheter of any one of Examples 1-8, wherein the microporous portion is configured such that at a balloon extraction pressure of at least 10 psi, the fluid exiting the balloon increases to an extraction flow rate of at least 5 ml/min.

Example 10 is the catheter of any one of Examples 1-9, wherein the balloon is comprised of at least one of Pebax, nylon, urethane, and polyester.

Example 11 is the catheter of any one of Examples 1-10, wherein the microporous portion is comprised of at least one of polytetrafluoroethylene, polypropylene, polycarbonate, Pebax, urethane, polyester and nylon.

Example 12 is the catheter of any one of Examples 6-11, wherein the microporous portion is integrated into either of a hub or a guidewire lumen of the tubular portion.

Example 13 is a method of manufacturing a catheter configured for ablation. The method including forming a microporous portion, forming a balloon including attaching a microporous portion, attaching conductors to the balloon, and attaching the balloon assembly to the catheter.

Example 14 is the method of Example 13, comprising wherein the step of attaching a microporous portion includes wherein the microporous portion comprises a plurality of apertures having a diameter that ranges from 0.05 microns to 50 microns.

Example 15 is the method of any one of Examples 13 and 14, wherein the plurality of apertures of the microporous portion is configured such that a flow of a substance may pass through the microporous portion at a flow rate that is greater than 0 mL/min and less than or equal to 1 mL/min at a nominal operating pressure.

Example 16 is a catheter for ablation. The catheter comprises a catheter shaft, a balloon at a distal end of the catheter shaft, and a microporous portion. The balloon is configured to support conductors and electrodes and is configured for containing a fluid. The microporous portion is coupled to the balloon, configured to allow the fluid to flow out of the balloon, and comprises a plurality of apertures configured to prevent air bubbles with a diameter larger than 50 microns from exiting the balloon.

Example 17 is the catheter of Example 16, wherein the microporous portion forms a portion of the balloon.

Example 18 is the catheter of Example 17, wherein the microporous portion of the balloon is a disc-shaped portion disposed at a distal portion of the balloon.

Example 19 is the catheter of Example 17, wherein the balloon includes a microporous strip portion comprising a plurality of apertures.

Example 20 is the catheter of Example 17, wherein the entire balloon is microporous with a plurality of apertures.

Example 21 is the catheter of Example 16, wherein the catheter further comprises a tubular portion coupled to the shaft and to the balloon, wherein the microporous portion is integrated into the tubular portion.

Example 22 is the catheter of Example 21, wherein the microporous portion is integrated into either of a hub or a guidewire lumen of the tubular portion.

Example 23 is the catheter of Example 16, wherein the plurality of apertures each have a diameter of between 0.05 microns and 50 microns.

Example 24 is the catheter of Example 16, wherein the microporous portion is configured such that at a balloon operating pressure of 1 psi, the fluid exits the balloon at an operating flow rate of less than or equal to 1 ml/min.

Example 25 is the catheter of Example 24, wherein the microporous portion is configured such that at a balloon extraction pressure of at least 10 psi, the fluid exiting the balloon increases to an extraction flow rate of at least 5 ml/min.

Example 26 is the catheter of Example 16, wherein the microporous portion is comprised of at least one of polytetrafluoroethylene, polypropylene, polycarbonate, Pebax, urethane, polyester and nylon.

Example 27 is a method of manufacturing a catheter configured for ablation. The method including forming a microporous portion, forming a balloon including attaching a microporous portion, attaching conductors to the balloon, and attaching the balloon assembly to the catheter.

Example 28 is the method of Example 27, comprising wherein the step of attaching a microporous portion includes wherein the microporous portion comprises a plurality of apertures having a diameter that ranges from 0.05 microns to 50 microns.

Example 29 is the method of Example 28, wherein the plurality of apertures of the microporous portion is configured such that a flow of a substance may pass through the microporous portion at a flow rate that is greater than 0 mL/min and less than or equal to 1 mL/min at a nominal operating pressure.

Example 30 is the method of Example 29, wherein the nominal operating pressure is 1 psi.

Example 31 is the method of Example 27, wherein the method further includes attaching electrodes to the balloon.

Example 32 is the method of Example 27, wherein the attaching of the microporous portion includes sealing the microporous portion onto the balloon.

Example 33 is a method of using a system for ablation comprising inserting a catheter comprising a shaft and a balloon into a patient, navigating and extending the catheter such that the catheter is in contact with a cardiac tissue of the patient, implementing ablation treatment through the electrodes to the cardiac tissue, and retracting the catheter such that a fluid passes through a plurality of openings of the balloon at a flow rate configured to prevent air bubbles with a diameter greater than a maximum value from passing through the balloon.

Example 34 is the method of Example 33, wherein during the retracting step, the fluid passes through a plurality of openings of the balloon at a flow rate that is greater than 0 mL/min.

Example 35 is the method of Example 33, wherein during the retracting step the flow rate of the fluid increases as an operating pressure of the balloon increases.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2A is a diagram illustrating the catheter, in accordance with embodiments of the subject matter of the disclosure.

FIG. 2B is a diagram illustrating the catheter, in accordance with embodiments of the subject matter of the disclosure.

FIG. 3 is a diagram illustrating a catheter adjacent cardiac tissue in the heart of a patient, in accordance with embodiments of the subject matter of the disclosure.

FIG. 4 is a method of manufacturing a catheter for a system for ablation, in accordance with embodiments of the subject matter of the disclosure.

FIG. 5 is a method of use of a catheter for a system of catheter ablation, in accordance with embodiments of the subject matter of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

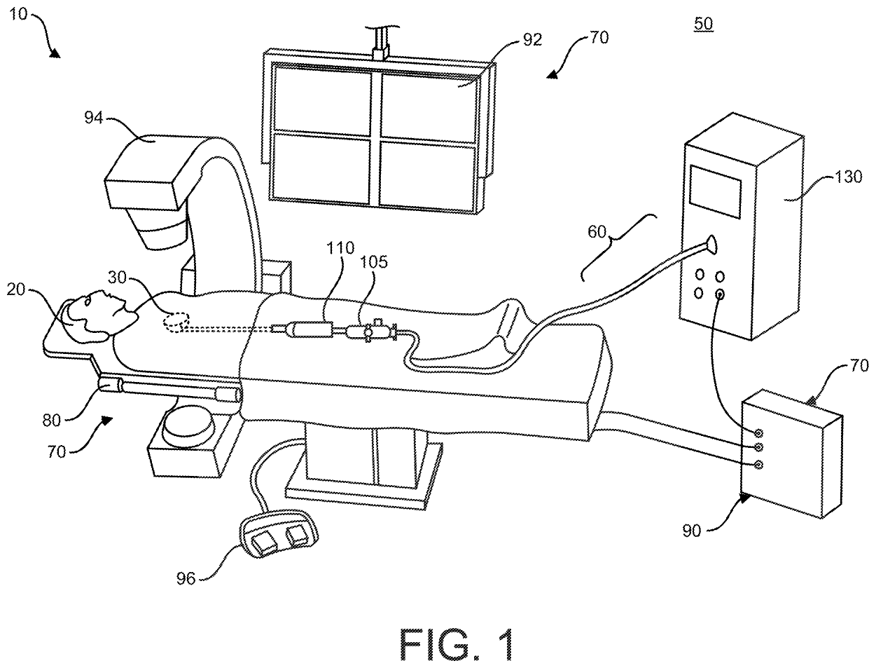

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes a catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

While the catheter system 60 could be used for a wide variety of procedures, including a variety of ablation procedures, in various embodiments described below, the catheter system 60 is an electroporation system. As will be apparent, aspects of the below description, while in the context of an electroporation system, will have applicability to other balloon catheter procedures, including other ablation procedures. The electroporation catheter system 60 includes an electroporation catheter 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the electroporation catheter system 60 is configured to deliver electric field energy to targeted tissue in the patient's heart 30 to create tissue apoptosis, rendering the tissue incapable of conducting electrical signals. Also, as will be described in greater detail below, the electroporation catheter system 60 is configured to generate, based on models of electric fields, graphical representations of the electric fields that can be produced using the electroporation catheter 105 and to overlay, on the display 92, the graphical representations of the electric fields on an anatomical map of the patient's heart to aid a user in planning ablation by irreversible electroporation using the electroporation catheter 105, prior to delivering energy. In embodiments, the electroporation catheter system 60 is configured to generate the graphical representations of the electric fields based on characteristics of the electroporation catheter 105 and the position of the electroporation catheter 105 in the patient 20, such as in the heart 30 of the patient 20. In embodiments, the electroporation catheter system 60 is configured to generate the graphical representations of the electric fields based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue.

The electroporation console 130 is configured to control functional aspects of the electroporation catheter system 60. In embodiments, the electroporation console 130 is configured to provide one or more of the following: modeling the electric fields that can be generated by the electroporation catheter 105, which often includes consideration of the physical characteristics of the electroporation catheter 105 including the electrodes and spatial relationships of the electrodes on the electroporation catheter 105; generating the graphical representations of the electric fields, which often includes consideration of the position of the electroporation catheter 105 in the patient 20 and characteristics of the surrounding tissue; and overlaying, on the display 92, the generated graphical representations on an anatomical map. In some embodiments, the electroporation control console 130 is configured to generate the anatomical map. In some embodiments, the EAM system 70 is configured to generate the anatomical map for display on the display 92.

In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web.

In embodiments, the introducer sheath 110 is operable to provide a delivery conduit through which the electroporation catheter 105 can be deployed to the specific target sites within the patient's heart 30.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 105, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure integrate the electroporation catheter system 60 with the EAM system 70 to allow graphical representations of the electric fields that can be produced by the electroporation catheter 105 to be visualized on an anatomical map of the patient and, in some embodiments, on an electro-anatomical map of the patient's heart. The integrated system of the present disclosure thus has the capability to enhance the efficiency of clinical workflows, including enhancement of planning the ablation of portions of the patient's heart by irreversible electroporation. Embodiments of the disclosure include generating the graphical representations of the electric fields that can be produced by the electroporation catheter 105, generating the anatomical maps, generating the electro-anatomical maps, and displaying information related to the location and electric field strengths of the electric fields that can be produced by the electroporation catheter 105.

In embodiments, the electroporation catheter 105 is a balloon catheter having electrodes situated inside or outside the balloon. The balloon is filled with a substance or fluid, such as saline. The catheter 105 includes a microporous portion that can be in the balloon, such as in the entirety of the balloon surface, a strip in the balloon, or embodied in a hub of the catheter 105 or the lumen that allows for passage of a guidewire. The microporous portion allows for the escape of the substance used to fill the balloon, but it does not allow large air bubbles to escape. Thus, any air that passes through the microporous portion would be effectively dissolved into the bloodstream.

While many of the figures and description herein describe the use of the catheter 105 in relation to ablation by electroporation, the catheter of the present disclosure may be applied to ablation processes in general, not limited to just electroporation. The application in this disclosure to electroporation is an exemplary embodiment of the present disclosure and is not meant to limit the application.

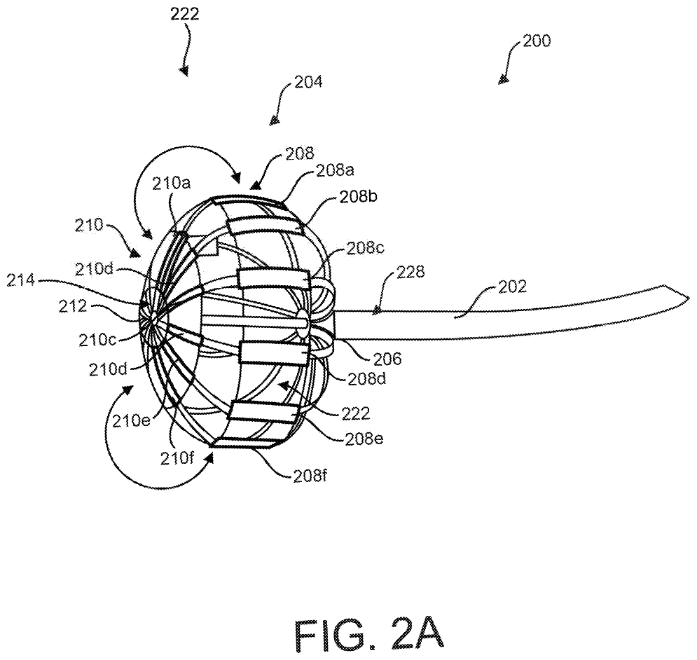

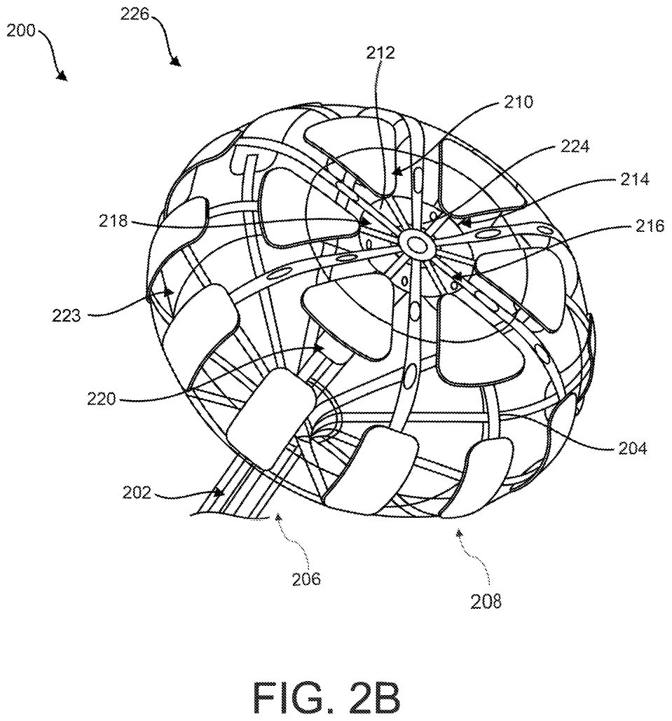

FIGS. 2A and 2B are diagrams illustrating a balloon catheter 200, in accordance with embodiments of the subject matter in the disclosure. In embodiments, the catheter 200 is used for ablation. This may include ablation by electroporation, including ablation by irreversible electroporation. In embodiments, the catheter 200 includes electrodes that are spaced apart from one another and configured to conduct electricity. Catheter characteristics are used to model electric fields that can be produced by the catheter. In embodiments, the characteristics used to model the electric fields can include: the type of catheter, such as a basket catheter that has a constant profile after being opened and a spline catheter that has a variable profile, which can be opened and closed by degree; the form factor of the catheter, such as a balloon catheter, a basket catheter, and a spline catheter; the number of electrodes; the inter-electrode spacing on the catheter; the spatial relationships and orientation of the electrodes, especially in relation to other electrodes on the same catheter; the type of material that the electrodes are made of; and the shape of the electrodes. In embodiments, the type of catheter and/or the form factor of the catheter includes catheters, such as linear ablation catheters and focal ablation catheters. Where, the type of catheter and/or the form factor of the catheter is not limited to those mentioned herein.

In embodiments, the catheter 200 is a balloon catheter having electrodes and conductors situated inside or outside the balloon. The balloon is filled with a substance or fluid, such as saline. In some embodiments, the catheter further comprises a catheter basket configured such that the balloon covers the catheter basket of the catheter 200. In other embodiments, the balloon may be disposed within the catheter basket. As shown, the catheter 200 includes a microporous portion on or in the balloon, such as in the entirety of the balloon surface, a disc-shaped portion of the balloon (e.g., near the distal tip), a strip in the balloon, or embodied in a hub of the catheter 200. The microporous portion allows for the escape of the substance used to fill the balloon, but it does not allow bubbles to escape, such that air that passes through the microporous portion would be effectively dissolved into the bloodstream and would not result in embolism.

FIG. 2A is a diagram illustrating the catheter 200, in accordance with embodiments of the subject matter of the disclosure. The catheter 200 includes a catheter shaft 202 and a balloon 222 attached to the catheter shaft 202 at a distal end 228 of the catheter 200. The balloon 222 includes a plurality of apertures 216. In some embodiments, the balloon 222 further includes a microporous tube portion comprising a plurality of apertures 216. In embodiments, the balloon 222 comprises a microporous strip portion comprising a plurality of apertures 216. In other embodiments, the entirety of the balloon 222 is comprised of apertures 216. In various embodiments, the catheter 200 further comprises a hub 212 that includes a microporous portion.

In various embodiments, the microporous portion 214 is constructed separately and attached to the main body of the balloon 222. In embodiments, the microporous portion is the microporous portion 214 of the balloon 222 previously described. The microporous portion 214 is configured such that a fluid that fills the balloon 222 can pass through the balloon 222 at a flow rate. Further, the microporous portion 214 is configured such that air bubbles with a diameter of 50 microns or greater are restricted from exiting the balloon 222. The balloon 222 is configured such that the fluid filling the balloon 222 causes the balloon 222 to inflate and expand.

In some embodiments, the microporous portion 214 is a disc-shaped portion. In other embodiments, the microporous portion 214 is a strip that is attached to the balloon 222. In embodiments, the microporous portion 216 is the entirety of the balloon 222 surface. The microporous portion 214 is comprised of a plurality of apertures 216 through which the fluid is able to pass through, as will be described further with reference to FIG. 2B. In embodiments, the balloon catheter 200 is configured for inflation in order to expand a passage or path in the body that may be occluded or narrowing. Further, in other embodiments, the catheter 200 includes electrodes and conductors and is configured for ablation.

FIG. 2B illustrates a perspective view of the catheter 200, including the balloon 222 disposed at a distal end 206 of the catheter shaft 202 and the microporous portion 214 coupled to the balloon 222. In embodiments, the balloon 222 is configured to contain a fluid 223 and configured for the support of electrode groups 208, 210 and conductors 204. In embodiments, the conductors 204 include a flex circuit on the balloon 222. In some embodiments, the balloon 222 comprises a hub 212. Further, in embodiments the hub 212 contains the microporous portion 214. In embodiments, the hub 212 is comprised of a microporous material. In embodiments, the microporous portion 216 of the hub 212 is a microporous material that is a sintered material or a rolled membrane. In other embodiments, the microporous portion of the hub 212 is comprised of expanded PTFE.

In embodiments, the microporous portion 214 includes the plurality of apertures 216. The plurality of apertures 216 is configured for the flow of a substance into the balloon 222 and out of the balloon 222. In this embodiment, the plurality of apertures 216 include a plurality of pores in the material. In embodiments, the microporous portion 214 is comprised of a disc-shaped portion 218. In embodiments, the microporous portion is formed in the tube portion 220. The tube portion 220 extends from a distal end 224 of the balloon 222 and extends to a position that is within a spacing enclosed by the balloon 222 or to a position in the catheter shaft 202. In some embodiments, the tube portion 220 includes both the hub 212 and a guidewire lumen used for introduction of a guidewire. In some embodiments, the electrode groups 208, 210 and/or conductors 204 placed on the balloon 222 are microporous and comprise the plurality of apertures 216. In embodiments, the electrodes of the electrode groups 208, 210 and/or conductors 204 include the microporous portion 214.

In embodiments, the microporous portion 214 is comprised of a microporous material that comprises the plurality of apertures 216. In embodiments, the microporous portion 214 is comprised of at least one of polytetrafluoroethylene, polypropylene, polycarbonate, Pebax, urethane, polyester and nylon. In embodiments, the balloon 222 is formed from at least one of Pebax, nylon, urethane, and polyester. In embodiments, the material of the balloon 222 is different than the material used for the microporous portion 214.

The plurality of apertures 216 allow for passage of a substance, such as saline, from the inside of the balloon 222 to the outside of the balloon 222, while preventing air bubbles that would otherwise be of danger to the patient from exiting the balloon 222. The air bubbles that are prevented from passing through the openings 216 include air bubbles that have a diameter exceeding a maximum value. In some embodiments, the diameter of the air bubbles that are restricted from exiting the plurality of apertures 216 is equal to or greater than 50 microns.

The substance flows from the balloon 222 at a flow rate that is influenced by the size of the apertures 216 or pore size in the balloon 222 and/or microporous portion 214. As the substance flows out of the microporous portion 214, there is an operating pressure produced within the catheter balloon 222. In some embodiments, this pressure may range from above 0 psi to 9 psi. In exemplary embodiments, the operating pressure is approximately 1 psi.

In embodiments, the size of each of the plurality of the apertures 216 ranges from 0.05 microns to 50 microns in diameter. In some embodiments, the size of the plurality of apertures 216 may range from 0.10 microns to 0.50 microns. In some embodiments, the size of the plurality of apertures 216 is 0.45 microns. The flow rate is influenced by at least the operating pressure of the balloon 222. In embodiments, the flow rate ranges from 0 mL/min to less than or equal to 1 mL/min while the balloon 222 operates with an operating pressure of 1 psi. In embodiments, the flow rate is 0.5 mL/min when the balloon operates at the operating pressure of 1 psi.

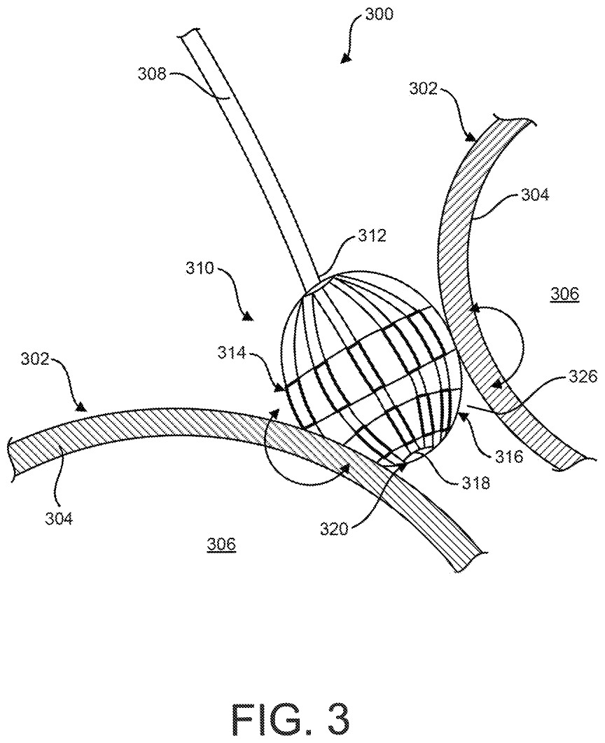

FIG. 3 is a diagram illustrating an electroporation catheter 300 adjacent cardiac tissue 302 in the heart of a patient, in accordance with embodiments of the subject matter of the disclosure. The cardiac tissue 302 includes endocardium tissue 304 and myocardium tissue 306, where at least some of the endocardium tissue 304 and myocardium tissue 306 may need to be ablated, such as by irreversible electroporation. In embodiments, the cardiac tissue 302 is part of the heart 30 of the patient 20.

The catheter 300 is suitable for performing ablation of the cardiac tissue 302, for example irreversible electroporation. The catheter 300 is not limited to irreversible electroporation and may be used for application of other ablation methods. The catheter 300 includes a catheter shaft 308 with a distal end 312 and a balloon 310 configured for supporting electrodes, such as those of a first group of electrodes 314 and a second group of electrodes 316, and conductors 332. The catheter 300 further includes a microporous portion 320 at a distal end 326 of the catheter 300. In embodiments, the microporous portion 320 of the catheter 300 may be the microporous portion 214 of catheter 200, and the balloon 310 may be the balloon 222 of the catheter 200.

In some embodiments, the balloon 310 includes a first group of electrodes 314 disposed at the circumference of the balloon 310 and a second group of electrodes 316 disposed adjacent the distal end 318 of the balloon 310. The catheter 300 may comprise variations of different electrode and conductor configurations other than the configuration described herein. In embodiments, each of the electrodes in the first group of electrodes 314 and each of the electrodes in the second group of electrodes 316 is configured to conduct electricity and to be operably connected to the electroporation console 130. In embodiments, one or more of the electrodes in the first group of electrodes 314 and the second group of electrodes 316 includes metal. In embodiments, the electroporation catheter 300 and the electrodes 314 and 316 are like the catheter 200 and the electrodes 208 and 210 previously described herein.

The catheter 300 and the electrodes of the first group and second group of electrodes 314 and 316 are or can be operably connected to the electroporation console 130, where the console 130 is configured to provide electric pulses to the electrodes 314 and 316 to produce electric fields that can ablate cardiac tissue 302 by irreversible electroporation. The dosing of the electric fields provided to the cardiac tissue 302 by the catheter 300, including the electric field strength and the length of time applied to the cardiac tissue 302, determines whether the cardiac tissue 302 is ablated.

For example, an electric field strength of about 400 volts per centimeter (V/cm) is considered large enough to ablate cardiac tissue 302, including myocardium tissue 306, in the heart by irreversible electroporation. While, electric field strengths of 1600 V/cm or more are needed to ablate or kill tissue, such as red blood cells, vascular smooth muscle, endothelium tissue, and nerve tissue, by irreversible electroporation. Also, reversible electroporation of cardiac tissue 302 in the heart can be accomplished with electric field strengths of 200-250 V/cm.

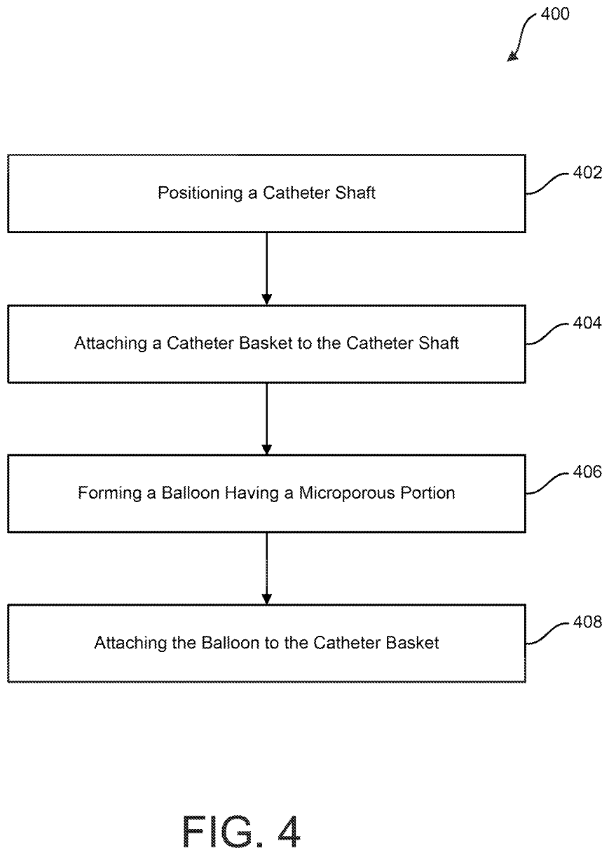

FIG. 4 is a method 400 of manufacturing a catheter 200 of a system for ablation. In embodiments, the catheter 200 may be for a system for ablation by electrophoresis. While the method is described in reference to the catheter 200 of FIG. 2A and FIG. 2B, the method also applies to the manufacture of the catheter 300 of FIG. 3. At block 402, the method includes forming a microporous portion 214. In embodiments, the microporous portion 214 is comprised of at least one of polytetrafluoroethylene, polypropylene, polycarbonate, Pebax, urethane, polyester and nylon. In embodiments, the microporous portion 214 includes a microporous disc-shaped portion that comprises the plurality of apertures 216. In some embodiments, the microporous portion 214 includes a tube portion comprising the plurality of apertures 216. In embodiments, the microporous portion 214 includes a microporous strip portion comprising the plurality of apertures 216. In other embodiments, the microporous portion 214 is configured to be a microporous portion configured to cover the entire balloon 222.

In embodiments, the microporous portion 214 of step 402 further includes wherein a plurality of apertures 216 of the microporous portion 214 have a diameter that may range from 0.05 microns to 50 microns. In various embodiments, the plurality of apertures 216 have a diameter that ranges from 0.05 microns to 1 micron. In embodiments, the plurality of apertures 216 of the microporous portion 214 of the balloon 222 are configured such that a flow of a substance exits the balloon 222 at a flow rate. In embodiments, the flow rate ranges from a value greater than 0 mL/min to a value less than or equal to 1 mL/min while the balloon is operating at a nominal operating pressure. In embodiments, this nominal operating pressure within the balloon 222 is approximately 1 psi.

At block 404, the method further comprises forming a balloon including attaching a microporous portion 214 to the balloon 222. In embodiments, the balloon 222 is formed from at least one of Pebax, nylon, urethane, and polyester. In embodiments, the attaching of the microporous portion 214 to the balloon 222 includes sealing the microporous portion 214 to the balloon 222.

At block 406, the method further includes attaching conductors 204 to the balloon 222. This step of attaching conductors 204 may include attaching the first and second group of electrodes 208, 210 to the balloon 222. In some embodiments, the conductor 204 is a conductive circuit in the form of a flex circuit. In these embodiments, the step 406 comprises wrapping the flex circuit onto the balloon 222. In embodiments, the flex circuit comprises the first and second electrode groups 208, 210 prior to the step of attachment.

At block 408, the method includes attaching a balloon assembly which includes the balloon 222 and attached elements such as the microporous portion 214, electrode groups 208, 210 and conductors 204, to the catheter 200. In embodiments, the catheter 200 comprises a catheter basket such that the balloon assembly is attached to cover the catheter basket. In other embodiments wherein a catheter basket is present, the balloon assembly is configured to be placed within the catheter basket.

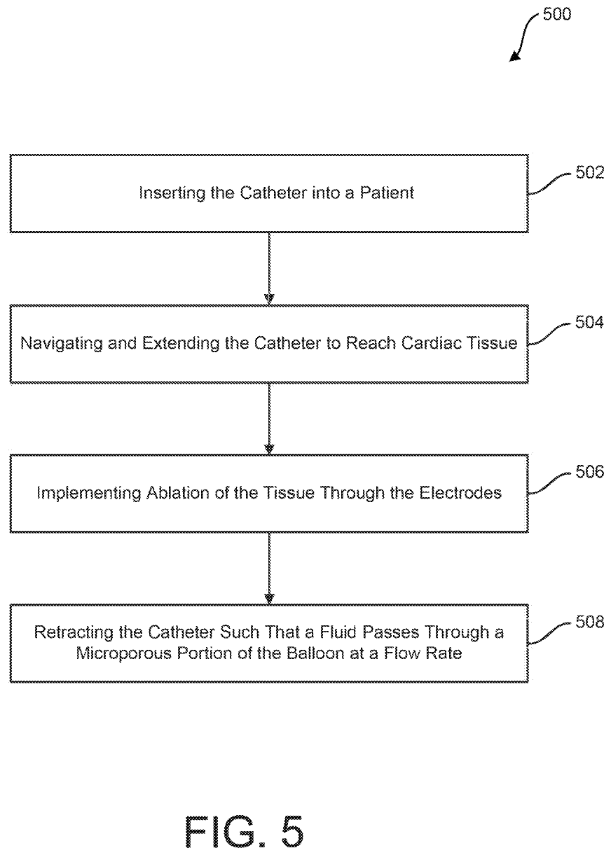

FIG. 5 is flowchart illustrating a method of use of a catheter of a system for ablation, in accordance with the present disclosure. The method is described in relation to catheter 200, however the catheter 300 may be used in the method as well. Also, in embodiments, the cardiac tissue 302 of FIG. 3 is configured to provide function in the method of use. Further, elements of the EAM system 70 can be configured to provide functions of the various steps of the method of use.

At 502, the method first includes inserting the catheter 200 into a patient. At 504, the method further includes the step of navigating and extending the catheter 200 to reach cardiac tissue 302, as illustrated in FIG. 3. In embodiments, the step further includes determining the location of the electrodes of the electrode groups 208, 210 in relation to the cardiac tissue 302 and determining the depth and surface area of the tissue that needs to be ablated. In embodiments, the step further comprises inflating the balloon 222 of the catheter 200 with a fluid. In embodiments, the fluid is saline.

At 506, the method 500 further includes implementing ablation treatment of the tissue through the electrodes 208 and/or 210. As described with reference to FIG. 3, the amount of voltage provided during the treatment may be controlled by the console 130 shown in FIG. 1. While described with reference to ablation by electrophoresis, the ablation treatment implemented may be that of different types of ablation treatment.

At 508, the method includes retracting the catheter 200 such that there is a fluid flow through a microporous portion 214 of the balloon 222 at a flow rate. During operation of the catheter 200, there is an operation pressure value within the balloon 222 which can influence the flow rate. In embodiments, the operating pressure ranges from a value greater than 0 psi to a value of 9 psi. In nominal operating conditions, the operating pressure may be 1 psi. In some embodiments, the flow rate ranges from a value that is greater than 0 mL/min and equal to or less than 1 mL/min while the operating pressure is approximately 1 psi. In embodiments, the flow rate will increase with an increasing operating pressure within the balloon 222. In embodiments, during the retraction step the operating pressure of the balloon 222 is 10 psi and the flow rate increases to at least 5 mL/min. The microporous portion 214 comprises a plurality of apertures 216 such that the fluid flows through. The plurality of apertures 216 of the microporous portion 214 is configured such that air bubbles having a diameter that exceeds a certain value are prevented from passing through the balloon 222 during the retracting step. In certain embodiments, the diameter value is the diameter of each of the plurality of apertures 216. In various embodiments, the diameter of the apertures 216 is 50 microns or less. In various embodiments, the diameter of the apertures 215 is between 0.05 and 0.5 microns. Further, in embodiments, at 508, the step includes wherein during the retracting of the catheter 200, the ability of the fluid to pass through the plurality of apertures 216 allows for the operating pressure to increase while maintaining the structural integrity of the balloon 222.

### Example 1

In an example consistent with the embodiments of the present disclosure, an operating catheter is described. The example is described with reference to the catheter 200 of FIG. 2A but may be applied to the catheter 300 of FIG. 3.

The catheter 200 includes a balloon 222 and a microporous portion 214 attached onto the balloon 200. In this example, the material forming the microporous portion 214 is comprised of nylon. The surface area of the microporous portion 214 that is used is 0.32 cm². The diameter of each of the plurality of apertures 216 of the microporous portion 214 is approximately 0.45 microns.

During operation, a fluid such as saline, flows into the balloon 222 of the catheter 200 and portions of the fluid are able to flow out of the plurality of apertures 216 of the microporous portion 214 at a flow rate. During operation of the catheter 200, there is an operating pressure within the balloon 222. This influences a flow rate of the saline out of the plurality of apertures 216. In this example, the operating pressure within the balloon 222 is about 1 psi and the flow rate of saline out of the balloon 222 is about 0.5 mL/min. A low flow rate of fluid through the balloon 222 is desired in order to minimize the amount of fluid disposed into the patient's bloodstream.

During retraction of the catheter 200 from the patient, the catheter 200 is withdrawn into the sheath and requires safe retraction of the balloon 222. The primary means of evacuating the saline from the catheter 200 during retraction is through the handle of the catheter 200. If any blockage occurs, such as if the saline is unable to evacuate through the handle, the operating pressure within the balloon 222 may increase. The microporous portion 214 of the catheter 200 is able to act as a secondary means of evacuating the saline. Due to the presence of the microporous portion 214 on the balloon 222, an increased operating pressure may cause an increased flow rate of fluid out of the balloon 222 through the microporous portion 214. In this example, an increased operating pressure of 20 psi during retraction of the balloon 222 could result in the flow rate of 10 mL/min. The microporous portion 214 allows for evacuation of flow to ensure the pressure does not increase to a value that may cause balloon rupture during retraction, which could otherwise result in dangerous air bubbles or balloon material being released into the patient.

The values of the present example are an exemplary embodiment of operation of the catheter 200. Other values of these parameters are possible and not limited to conditions the values within the ranges described in the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

1. A catheter for ablation, the catheter comprising:
a catheter shaft;
a balloon disposed at a distal end of the catheter shaft, the balloon configured to support conductors and electrodes and further configured to contain a fluid;
a microporous portion coupled to the balloon and configured to allow the fluid to flow out of the balloon; and
wherein the microporous portion includes a plurality of apertures configured to prevent air bubbles with a diameter larger than 50 microns from exiting the balloon.

2. The catheter of claim 1, wherein the microporous portion forms a portion of the balloon.

3. The catheter of claim 2, wherein the microporous portion of the balloon is a disc-shaped portion disposed at a distal portion of the balloon.

4. The catheter of claim 2, wherein the balloon includes a microporous strip portion comprising a plurality of apertures.

5. The catheter of claim 2, wherein the entire balloon is microporous with a plurality of apertures.

6. The catheter of claim 1 further comprising a tubular portion coupled to the shaft and to the balloon, wherein the microporous portion is integrated into the tubular portion.

7. The catheter of claim 6, wherein the microporous portion is integrated into either of a hub or a guidewire lumen of the tubular portion

8. The catheter of claim 1, wherein the plurality of apertures each have a diameter of between 0.05 microns and 50 microns.

9. The catheter of claim 1, wherein the microporous portion is configured such that at a balloon operating pressure of 1 psi, the fluid exits the balloon at an operating flow rate of less than or equal to 1 ml/min.

10. The catheter of claim 9 wherein the microporous portion is configured such that at a balloon extraction pressure of at least 10 psi, the fluid exiting the balloon increases to an extraction flow rate of at least 5 ml/min.

11. The catheter of claim 1, wherein the microporous portion is comprised of at least one of polytetrafluoroethylene, polypropylene, polycarbonate, Pebax, urethane, polyester and nylon.

12. A method to manufacture a catheter configured for ablation comprising the steps of:
forming a microporous portion
forming a balloon, including attaching a microporous portion;
attaching conductors to the balloon; and
attaching the balloon assembly to the catheter.

13. The method of claim 12, wherein the step of attaching a microporous portion includes wherein the microporous portion comprises a plurality of apertures having a diameter that ranges from 0.05 microns to 50 microns.

14. The method of claim 13, wherein the plurality of apertures of the microporous portion is configured such that a flow of a substance may pass through the microporous portion at a flow rate that is greater than 0 mL/min and less than or equal to 1 mL/min at a nominal operating pressure.

15. The method of claim 14, wherein the nominal operating pressure is 1 psi.

16. The method of claim 12, wherein the method further includes attaching electrodes to the balloon.

17. The method of claim 12, wherein the attaching of the microporous portion includes sealing the microporous portion onto the balloon.

18. A method of using a system for ablation comprising:
inserting a catheter comprising a shaft and a balloon into a patient;
navigating and extending the catheter such that the catheter is in contact with a cardiac tissue of the patient;
implementing ablation treatment through the electrodes to the cardiac tissue; and
retracting the catheter such that a fluid passes through a plurality of openings of the balloon at a flow rate configured to prevent air bubbles with a diameter greater than a maximum value from passing through the balloon.

19. The method of use of claim 18, wherein during the retracting step, the fluid passes through a plurality of openings of the balloon at a flow rate that is greater than 0 mL/min.

20. The method of use of claim 18, wherein during the retracting step the flow rate of the fluid increases as an operating pressure of the balloon increases.
